(19) United States
(12) Patent Application Publication
(10) Pub. No.: US 2024/0066488 A1
(43) Pub. Date: Feb. 29, 2024

(54) MULTISTAGE REACTOR FOR PERFORMING EXOTHERMIC EQUILIBRIUM REACTIONS

(71) Applicant: L'Air Liquide, Société Anonyme pour l'Etude et l'Exploitation des Procédés Georges Claude, Paris (FR)

(72) Inventors: Michael STROZYK, Offenbach (DE); Frank CASTILLO-WELTER, Friedrichsdorf (DE); Tobias OELMANN, Bad Vilbel (DE); Timm SCHUHMANN, Bensheim (DE)

(21) Appl. No.: 18/237,960

(22) Filed: Aug. 25, 2023

(30) Foreign Application Priority Data
Aug. 26, 2022 (EP) 22192298.2

(51) Int. Cl. B01J 8/04; B01D 53/00

(52) U.S. Cl. CPC B01J 8/0446; B01D 53/002; B01J 8/0496; B01J 2208/00115; B01J 2208/021

(57) ABSTRACT

The invention relates to a reactor for performing exothermic equilibrium reactions, especially for producing methanol from synthesis gas in a multistage synthesis with intermediate condensation of the reaction product. The reactor according to the invention has a reactor shell and a multitude of series-connected and mutually fluid-connected reactor cells disposed within the reactor shell, where each of the reactor cells includes a reaction apparatus, a cooling-down apparatus and a phase separation apparatus as reactor cell elements. The reactor has a multitude of reactor planes disposed in a mutually parallel arrangement within the reactor shell, where reactor cell elements of the same kind are disposed in the same reactor plane. The inventive arrangement of the reactor cell elements enables the building of a compact reactor and reduces material stresses within the reactor by the avoidance of large temperature differences within the reactor shell.

### CROSS REFERENCE TO RELATED APPLICATIONS

This application claims the benefit of priority under 35 U.S.C. § 119 (a) and (b) to European Patent Application No. 22192298.2, filed Aug. 26, 2022, the entire con-tents of which are incorporated herein by reference.

### BACKGROUND

### Technical Field of the Invention

The invention relates to a reactor, especially a multistage reactor for performing exothermic equilibrium reactions with intermediate condensation of the reaction product. The invention further relates to the use of the reactor according to the invention for the production of methanol.

### Prior Art

EP 3 401 299 A1 and EP 3 401 300 describe the principle of a multistage reactor with intermediate condensation, for example for the production of methanol with intermediate condensation of the crude methanol (methanol, water and impurities). According to this principle, multiple reactor cells are disposed within a common shell, with each of the reactor cells comprising at least the actual reaction zone, a cooling-down zone, a separation zone, and means of discharging the separated reaction product and of discharging the unconverted feed gas.

For the spatial arrangement of the reactor cells within the reactor shell, EP 3 401 299 A1 proposes exclusively a successive arrangement of the reactor cells in a vertical (upright) or horizontal (recumbent) manner.

However, simply arranging reactor cells in succession results in disadvantageous reactor shell dimensions.

In one example, a length of about 7 metres per stage is found for a three-stage reactor, assuming that the catalyst bed length of the reaction zone is 4 metres, the heat exchanger beyond that has a length of about 1.5 metres, and the condenser and separator together occupy a length of 1.5 metres. These reactor cells stacked one on top of another already give rise to a total length of 21 metres.

Particularly in the case of reactors of comparatively small capacity with correspondingly small reactor shell diameters, an unfavourable length-diameter ratio is found here. An additional steel framework is required in order to ensure the mechanical stability of such a slim apparatus. It is also difficult to handle the catalyst, particularly in the lower reactor cells. The conducting of the gas within and between the reactor cells is complex and requires complicated internals, piping and flow channels. When thermoplates are used as heat exchanger structure (also referred to as pillow plate heat transferrers), respective individual thermoplate assemblies have to be manufactured with a header and collector and installed in the shell, which leads to higher production costs. Temperature control is also not optimal in such a configuration, since, for example, a hot reaction zone of a second reactor cell is in the direct proximity of a cold deposition zone of the upstream first reactor cell. This requires a correspondingly inconvenient insulation. Performance in the separation zone can also be impaired by heating of the liquid phase in the separator, which leads to losses in the product yield. The associated thermal stresses in the metal used also require a complex design that has to be taken into account in the construction of the apparatus.

Alternatively, the reaction zone, the cooling-down zone and the separation zone may be arranged horizontally in a common reactor shell. In relation to the total length of an apparatus, in accordance with the above example, it is possible here to achieve savings with regard to the total length of the recumbent apparatus if the process gas flows in from the top downward, at right angles to the horizontal axis of the recumbent apparatus. In accordance with the above example, this case would result in a total length of about 5.70 metres. Dividing the reaction zones can reduce the shell diameter, but this leads to a more complicated process gas regime. A further approach to a solution could lie in horizontal flow toward the reactor bed. The disadvantage here, however, is that bypass flow caused by catalyst shrinkage is technically difficult to control. Moreover, over the lifetime of the catalyst, it can be assumed that the catalyst bed will become successively compacted in the lower region of the bed, such that increasing maldistribution of the product gas over the catalyst bed will arise over the lifetime of the catalyst in the case of horizontal inflow. As in the above example with vertical arrangement, even in the case of a horizontal arrangement of the reactor cells in space as successive rows, the temperature regime is afflicted by drawbacks and the process gas regime is complicated.

US 2018/0221842 A1 relates to a multistage reactor and to a system for production of methanol from synthesis gas. The reactor contains a shell-and-tube reactor divided into multiple vertical isolated compartments at its upper and lower ends. The corresponding compartments and tubes form one stage of the reactor. The crude synthesis gas is fed to the first stage, and the unconverted synthesis gas from the first stage is fed to the second stage downstream. Between the individual stages, the product, methanol and water, is removed from the reaction mixture before the unconverted synthesis gas is guided into the downstream stage. The heat exchangers and separators are disposed outside the reactor shell, which leads to a complicated arrangement of conduits and a complex process regime.

### SUMMARY

It is an object of the present invention to at least partially overcome the aforementioned disadvantages of the prior art.

In particular, it is an object of the present invention to provide a reactor that enables the arrangement of a multitude of reaction apparatuses, cooling-down apparatuses and phase separation apparatuses within a compact housing.

It is a further object of the present invention to provide a reactor that enables the arrangement of a multitude of reaction apparatuses, cooling-down apparatuses and phase separation apparatuses within a common reactor shell.

It is a further object of the present invention to provide a reactor which, in the case of a multitude of reaction apparatuses and phase separation apparatuses, avoids the arrangement of a reaction apparatus and a phase separation apparatus directly alongside one another.

It is a further object of the present invention to provide a reactor which, in the case of a multitude of reaction apparatuses, cooling-down apparatuses and phase separation apparatuses, enables a homogenized temperature profile over the entire length of the reactor, such that material stresses are avoided and less expensive materials can be used with regard to the reactor components.

A contribution to the at least partial achievement of at least one of the above objects is made by the independent claims. The dependent claims provide preferred embodiments which contribute to the at least partial achievement of at least one of the objects. Preferred embodiments of constituents of one category according to the invention are, where relevant, likewise preferred for identically named or corresponding constituents of a respective other category according to the invention.

The terms “having”, “comprising” or “containing” etc. do not preclude the possible presence of further elements, ingredients etc. The indefinite article “a” does not preclude possible presence of a plurality.

In a first aspect of the invention, a reactor for performing exothermic equilibrium reactions is proposed, in which a feed gas mixture and/or a residual gas mixture is/are at least partly convertible over a solid catalyst, wherein a product gas mixture and a residual gas mixture are obtainable, wherein the product gas mixture contains a liquid reaction product which is condensable at reaction pressure and below the reaction temperature, having
a reactor shell and a multitude of series-connected and mutually fluid-connected reactor cells disposed within the reactor shell, wherein
each reactor cell comprises the following series-connected and mutually fluid-connected reactor cell elements:
   a reaction apparatus configured for converting the feed gas mixture and/or the residual gas mixture to the product gas mixture, having the solid catalyst and a cooling apparatus which is in a heat-exchanging relationship with the solid catalyst and through which a cooling medium can flow;
   a cooling-down apparatus configured for cooling down the product gas mixture exiting from the reaction apparatus;
   a phase separation apparatus configured for condensing and separating the liquid reaction product from the cooled-down product gas mixture, where the condensed liquid phase contains the reaction product, and the gaseous phase the residual gas mixture;
and wherein each reactor cell comprises
   means of discharging the reaction product from the reactor cell, and
   means of discharging the residual gas mixture from the reactor cell, and
   means of feeding the residual gas mixture to a downstream reactor cell, if present;
and wherein the reactor has
   a multitude of reactor planes disposed in a mutually parallel arrangement within the reactor shell, where reactor cell elements of the same kind are disposed in the same reactor plane.

The reactor according to the invention is configured to perform exothermic equilibrium reactions, especially to perform the synthesis of methanol on an industrial scale from synthesis gas. The feed gas mixture is especially a synthesis gas mixture, where the synthesis gas mixture includes at least hydrogen as a first synthesis gas, and at least carbon monoxide or carbon dioxide as a second synthesis gas. In particular, the feed gas mixture includes hydrogen as first reactant gas, carbon monoxide as second reactant gas, and carbon dioxide as third reactant gas.

At least some of the reactor cell elements are connected to the reactor shell. It is not necessarily the case that each of the reactor cell elements is mechanically connected to the reactor shell. In one embodiment, reactor cell elements are connected to one another by mechanical connecting elements, such that there is no requirement for a mechanical connection to the reactor shell for each of the reactor cell elements. The mechanical connection may be a mechanical support structure, for example in the form of a means of suspension or bracing.

The product gas mixture is formed at elevated temperature (reaction temperature) and at elevated pressure. If the reaction product is methanol and water, for example, the product gas mixture is especially formed at temperatures of 180° C. to 250° C. and at pressures of 30 bar to 120 bar, especially at 40 bar to 90 bar. With regard to the temperature, the working temperature range of the catalyst used should be noted, and the temperature should be no higher or lower. For the methanol synthesis, in general, solid copper-based catalysts known to the person skilled in the art are used.

The product gas mixture especially contains methanol and water and unavoidable by-products such as ethers, esters, higher alcohols and ketones, and the mixture of methanol, water and by-products is also referred to as crude methanol. At least methanol and water in this case are the liquid reaction products that are condensable at reaction pressure and below the particular reaction temperature. Since the formation of methanol and water from synthesis gas is an equilibrium reaction in which a thermodynamic equilibrium is established, a proportion of the feed gas mixture, especially synthesis gas mixture, is not converted to methanol and water. This unconverted proportion forms the residual gas mixture.

The reactor according to the invention has a reactor shell and a multitude of series-connected and mutually fluid-connected reactor cells disposed within the reactor shell. What is meant by “multitude” in this connection is that the reactor comprises at least two reactor cells. Each reactor cell forms a reaction stage of a multistage synthesis. What is meant by multistage synthesis is that, in each stage of the multistage synthesis, product gas mixture is formed from feed gas mixture and/or residual gas mixture over the solid catalyst, and the desired liquid reaction product is at least partly separated from the resultant product gas mixture. The reactor cells are arranged in succession, meaning that the second reactor cell is disposed downstream of the first reactor cell in gas flow direction, the third reactor cell, if present, is disposed downstream of the second reactor cell in gas flow direction, and so forth. The reactor cells are mutually fluid-connected. This means more particularly that a gas mixture, especially residual gas mixture, which is discharged from a reactor cell is subsequently introduced into the respective reactor cell downstream in gas flow direction.

The product gas mixture is cooled down in the first reactor cell to such an extent that it can be at least partly condensed and hence the liquid reaction product can be discharged from the first reactor cell. The residual gas mixture is at least partly introduced into the reactor cell downstream of the first reactor cell in gas flow direction, in order that product gas mixture can again be formed from residual gas mixture in the second reactor cell over the solid catalyst in the second reactor cell. In one embodiment, it is also possible to mix a proportion of the gas mixture (“fresh gas”) into the residual gas mixture which is introduced into the second reaction cell. Product gas mixture from the second reactor cell is again cooled down to such an extent that the reaction product is at least partly condensed out of it and can be discharged in liquid form from the second reactor cell. If a third reactor cell is present, the residual gas mixture remaining in the second reactor cell and optionally a proportion of the gas mixture are fed into the third reactor cell, and so forth.

Each of the multitude of reactor cells comprises a reaction apparatus, a cooling-down apparatus and a phase separation apparatus. The reaction apparatus, the cooling-down apparatus and the phase separation apparatus in the context of the reactor of the invention are different kinds of reactor cell elements that are each disposed within a reactor cell. Each reactor cell comprises at least a reaction apparatus, a cooling-down apparatus and a phase separation apparatus.

The reaction apparatus is an apparatus configured for the formation of the product gas mixture from the feed gas mixture and/or residual gas mixture over the solid catalyst. The reaction apparatus has a cooling apparatus in a heat-exchanging relationship with the solid catalyst. The cooling apparatus especially serves to control the temperature of the exothermic equilibrium reaction, i.e. for removal of heat of reaction. The cooling apparatus is especially configured such that a particular fixed maximum temperature in the catalyst bed of the fixed catalyst is not exceeded. In one embodiment, the cooling medium is boiling boiler feed water, and the cooling in the reaction apparatus is effected by the principle of evaporative cooling. The boiling boiler feed water forms steam, which can be utilized for the purpose of energy recovery, for example as export steam. The solid catalyst may be a catalyst bed based on catalyst particles (pellets) or a structured catalyst, for example with monolithic structure.

Each of the reactor cells further comprises at least one cooling-down apparatus. The cooling-down apparatus serves to cool down the product gas mixture exiting from the reaction apparatus. The product gas mixture is preferably cooled down in the cooling-down apparatus only to such an extent that there is still no condensation of the liquid reaction product, meaning that the reaction product remains in the gas phase within the cooling-down apparatus. The reaction product is especially a mixture of methanol and water, and optionally further condensable and/or non-condensable (unwanted) by-products. Within a reactor cell, the cooling-down apparatus is disposed downstream of the reaction apparatus of the same reactor cell in gas flow direction. The cooling-down apparatus preferably has a cooling apparatus in a heat-exchanging relationship with the product gas mixture exiting from the reaction apparatus. The cooling apparatus is especially an indirect heat exchanger, wherein possible cooling media are a gas or a liquid. The cooling medium is preferably the feed gas mixture or residual gas mixture. If it is the feed gas mixture, this is preferably fed to the cooling-down apparatus of the first reactor cell as cooling medium. If it is the residual gas mixture, this is preferably fed to a cooling-down apparatus of a reactor cell disposed downstream of the first reactor cell in gas flow direction, i.e. to the second and/or third reactor cell, and so forth. In particular, this residual gas mixture is drawn off from the phase separation apparatus of the reactor cell upstream of the respective reactor cell, i.e. from the phase separation apparatus of the first reactor cell, in order to feed it to the cooling-down apparatus of the second reactor cell, and/or from the phase separation apparatus of the second reactor cell, in order to feed it to the cooling-down apparatus of the third reactor cell, and so forth.

Each of the reactor cells further comprises at least one phase separation apparatus. The phase separation apparatus serves to further cool down the product gas mixture that has already been precooled in the cooling-down apparatus, such that the reaction product is condensed and is obtained in liquid form. The phase separation apparatus is thus configured such that a condensed liquid phase and a gaseous phase are obtained. In addition, the phase separation apparatus serves to separate the liquid phase from the gaseous phase, especially the liquid reaction product from the further constituents, i.e. non-liquefied constituents, meaning those that have not been condensed or are not condensable in the phase separation apparatus. If the feed gas mixture is a synthesis gas mixture and the target product is methanol, the liquid phase will contain, as condensed liquid reaction product, methanol and water, and (unwanted) by-products that are condensable under the respective conditions. The gaseous phase contains the residual gas mixture, i.e. unconverted synthesis gas, and by-products that are uncondensable under the respective conditions. Uncondensable by-products also include constituents that are already present in the feed gas mixture and are inert under the conditions of methanol synthesis, meaning that they are converted neither to the methanol target product nor to a by-product. Examples of this type of by-products are methanol and nitrogen.

Within a reactor cell, the phase separation apparatus is disposed downstream of the cooling-down apparatus of the same reactor cell in gas flow direction. The phase separation apparatus preferably has a cooling apparatus in a heat-exchanging relationship with the product gas mixture exiting from the cooling-down apparatus. The cooling apparatus is especially an indirect heat exchanger, wherein a possible cooling medium is preferably a liquid, especially cooling water. The phase separation apparatus also preferably further comprises an apparatus for phase separation, especially a gas-liquid separator.

The reactor according to the invention has a multitude of reactor cells, where these reactor cells are arranged in series. This means that the first reactor cell is followed in gas flow direction by a second reactor cell, the second reactor cell is optionally followed by a third reactor cell, and so forth. If the reactor has a number N of reactor cells, the first reactor cell is the [N-(N-1)]th reactor cell, the second reactor cell is the [N-(N-2)]th reactor cell, and so forth, and the last reactor cell is the [N-(N-N)]th or Nth reactor cell. Each of the reactor cells has at least a reaction apparatus, a cooling-down apparatus, and a phase separation apparatus, where the cooling-down apparatus in each case is disposed downstream of the reaction apparatus in gas flow direction and the phase separation apparatus in each case downstream of the cooling-down apparatus of a reactor cell. Because of the arrangement of the reactor cells in series, the reaction apparatus of a second reactor cell is disposed downstream of the phase separation apparatus of a first reactor cell in gas flow direction. The reaction apparatus of a third reactor cell is disposed downstream of the phase separation apparatus of a second reactor cell, and so forth. If a reactor according to the invention, for example, contains a total of three reactor cells, the arrangement in respect of the sequence in gas flow direction is as follows, assuming that each reactor cell has a reaction apparatus, a cooling-down apparatus and a phase separation apparatus, and each of these reactor cell elements bears the number of the reactor cell as index (in brackets):
reaction apparatus (1)→cooling-down apparatus (1)→phase separation apparatus (1)→reaction apparatus (2)→cooling-down apparatus (2)→phase separation apparatus (2)→reaction apparatus (3)→cooling-down apparatus (3)→phase separation apparatus (3).

If the reactor has a number N of reactor cells, the sequence is generally as follows, assuming that each reactor cell has a reaction apparatus, a cooling-down apparatus and a phase separation apparatus:
reaction apparatus ([N-(N-1)])→cooling-down apparatus ([N-(N-1)]) phase separation apparatus ([N-(N-1)])→reaction apparatus ([N-(N-2)])→cooling-down apparatus ([N-(N-2)])→phase separation apparatus ([N-(N-2)])→[ . . . ]
reaction apparatus (([N-(N-N)]) cooling-down apparatus ([N-(N-N)])→phase separation apparatus ([N-(N-N)]).

Each of the reactor cells comprises means of discharging the reaction product from the respective reactor cell. In particular, the reactor according to the invention comprises means of discharging the liquid reaction product from the phase separation apparatus of the respective reactor cell. The liquid reaction product discharged from each of the reactor cells, which is especially crude methanol, is then sent to a further workup, for example rectification. In one embodiment, the streams of liquid reaction product that are discharged from each of the reactor cells are combined and subjected collectively to a further workup.

Each of the reactor cells further comprises means of discharging the residual gas mixture from the respective reactor cell. In particular, the reactor according to the invention comprises means of discharging the residual gas mixture from the phase separation apparatus of the respective reactor cell. The residual gas mixture discharged from each of the reactor cells, in the case of methanol synthesis, especially contains unconverted synthesis gas mixture, and hence unconverted hydrogen and carbon monoxide and/or carbon dioxide. In addition, the residual gas mixture contains uncondensable by-products, for example methane and nitrogen.

In addition, each of the reactor cells comprises means of feeding the residual gas mixture to a downstream reactor cell, if there is a reactor cell disposed downstream. What is meant by “disposed downstream” in this context is that the reactor cell is disposed downstream of the previous reactor cell in gas flow direction. In particular, each of the reactor cells comprises means of feeding the residual gas mixture to a downstream reaction apparatus, if there is a reaction apparatus disposed downstream. If the reactor cell is the last reactor cell of the N reactor cells, i.e. the Nth reactor cell, there is no downstream reactor cell. Residual gas mixture which is still unconverted at this point can optionally be recycled back to the first reactor cell. In this embodiment, the last or Nth reactor cell has means of feeding the residual gas mixture to the first or [N-(N-1)]th reactor cell, especially to the reaction apparatus of the first or [N-(N-1)]th reactor cell. In one embodiment, a purge gas is drawn off from the residual gas discharged from the last reactor cell, in order to prevent the enrichment of inert components in the reactor.

According to the invention, the reactor has a multitude of reactor planes disposed in a mutually parallel arrangement within the reactor shell, where reactor cell elements of the same kind are disposed in the same reactor plane. In particular, a multitude of reactor cell elements of the same type is disposed in the same reactor plane. For example, a multitude of reaction apparatuses is disposed in one and the same reactor plane, a multitude of cooling-down apparatuses is disposed in a further reactor plane, and a multitude of phase separation apparatuses is disposed in a further reactor plane. A plane is especially understood to mean a surface that extends over a cross-sectional area of the reactor of the invention and is arranged perpendicular to a principal axis of the reactor. The reactor has a multitude of such reactor planes that are arranged parallel to one another. If the reactor according to the invention extends, for example, in a vertical principal direction and hence has a vertical principal axis, the reactor planes will extend perpendicular to the main axis of the reactor in horizontal direction and will be in a parallel, mutually superposed arrangement. A reactor plane is not necessarily something physical, for example a plate. A “reactor plane” in the context of the present invention is also understood to mean, but is not limited to, a virtual reactor plane, which means in consequence that the multitude of the respective reactor cell elements of one kind, according to the above example, is disposed essentially at the same height, i.e. in a plane. According to the invention, a reactor plane may must be understood to mean either a physical or virtual reactor plane.

The arrangement according to the invention gives rise, for example, to the advantage that the reaction apparatuses are fundamentally arranged spaced apart from the phase separation apparatuses, such that heating of the media in the phase separation apparatuses by the reaction apparatuses is fundamentally avoided. Since a cooling-down apparatus is disposed downstream of a reaction apparatus in gas flow direction, and a phase separation apparatus is disposed downstream of a cooling-down apparatus, the reaction apparatuses in such a case would be present in a first plane, the cooling-down apparatuses in a second plane, and the phase separation apparatuses in a third plane. This ensures spatial separation of the reaction apparatuses and the phase separation apparatuses.

The inventive arrangement of the reactor cell elements also results in the advantage of a short pressure shell length compared to an arrangement in which all reactor cell elements are arranged along a common principal axis, it being material whether the reactor is in a recumbent or upright arrangement. According to the invention, the ratio of length to diameter of the reactor in each case is smaller and hence the dimensions are more favourable.

Moreover, the arrangement according to the invention makes it possible for the different reactor cell elements to be arranged in a sequence from “hot to cold”, in which case the temperatures in the reactor cell that exist in a reactor plane would be essentially the same. This results in occurrence of low to zero thermal stresses in radial direction of the reactor. For example, from one reactor plane to the next, there may be an arrangement first of the reaction apparatuses (hot), then of the cooling-down apparatuses (colder), and then of the phase separation apparatuses (even colder). Thus, even along the principal axis of the reactor, for example in vertical direction, in the case of corresponding arrangement of the reactor cell elements, there is no occurrence of extreme temperature jumps as would be the case, for example, in a series arrangement of a reaction apparatus and a phase separation apparatus. Such a disadvantageous arrangement can be avoided by the inventive arrangement of the reactor cell elements.

The arrangement according to the invention also enables a modular construction of the reactor that permits an extension to include further reactor cells at a later juncture.

A preferred embodiment of the reactor according to the invention is characterized in that exclusively one kind of reactor cell element is disposed in each of the reactor planes.

For example, solely a multitude of reaction apparatuses is disposed in a first reactor plane, solely a multitude of cooling-down apparatuses in a second reactor plane, and solely a multitude of phase separation apparatuses in a third reactor plane. This enables a reactor design of maximum compactness, irrespective of how many reactor cells and, accordingly, how many reactor cell elements the reactor cell comprises.

A preferred embodiment of the reactor according to the invention is characterized in that the number of reactor planes present corresponds at least to the number of types of reactor cell element present.

If the reactor according to the invention has, for example, three different kinds of reactor cell element, especially a reaction apparatus, a cooling-down apparatus and a phase separation apparatus, the number of reactor planes present corresponds at least to the number of kinds of reactor cell elements present, i.e. to the number three or more, preferably three.

The reactor according to the invention is therefore preferably characterized in that the number of reactor planes present corresponds to the number of types of reactor cell element present.

A preferred embodiment of the reactor according to the invention is characterized in that the reactor cells are in a vertical arrangement with respect to the perpendicular imparted by gravity, such that streams are conductable from the bottom upward and from the top downward within and between the reactor cell elements.

The effect of the vertical arrangement of the reactor cells within the reactor shell is that the overall reactor extends in vertical direction on account of the successive arrangement of different reactor cell elements of a reactor cell as the principal extent. At the same time, the reactor has less of a horizontal extent. As a result, a comparatively small setup area is required for the overall reactor, even if there are a multitude of reactor cells. At the same time, the overall reactor is not excessively tall since all reactor cell elements of the same kind are each disposed in a common reactor plane.

A preferred embodiment of the reactor according to the invention is characterized in that the reactor cell elements are arranged within the reactor shell according to a two-dimensional matrix with a number y of columns and a number x of rows, where the number y corresponds to columns of the number of reactor cells, and the number x corresponds to rows of the number of different types of reactor cell element, where one column in the matrix comprises one reactor cell of each of the different types of reactor cell element, and one row in the matrix comprises a reactor plane having a number of reactor cell elements of the same type.

An arrangement of the reactor cell elements in the manner of a two-dimensional matrix achieves maximum minimization of the volume taken up by the reactor.

A preferred embodiment of the reactor according to the invention is characterized in that the reactor planes are arranged parallel to one another and successively from the top downward.

In particular, the reactor planes in this embodiment extend in a horizontal direction, such that the arrangement of the reactor planes should be regarded as a stack of reactor planes in a horizontal arrangement, where the distances between the individual reactor planes correspond essentially to the height taken up by the respective reactor cell element.

A preferred embodiment of the reactor according to the invention is characterized in that the reactor has a first end and a second end, with reactor cell elements in the reactor planes arranged spatially according to the following sequence from the first end in the direction of the second end: reaction apparatus, cooling-down apparatus, phase separation apparatus.

For maximum simplification of the flow regime and for maximum spacing of the phase separation apparatuses from the reaction apparatuses, the individual reactor cell elements within a reactor cell are arranged according to the above sequence. Such reactor thus comprises a total of three reactor planes, where the sequence of arrangement of the reactor cell elements are reactor cell from the first end toward the second end is as follows: reaction apparatus, cooling-down apparatus, phase separation apparatus. The terms “first end” and “second end” should be understood in relation to spatial arrangement of the reactor cell elements within the reactor shell, not to the flow regime within the reactor.

A preferred embodiment of the reactor according to the invention is characterized in that each phase separation apparatus comprises a condensation apparatus and a separation apparatus, where the condensation apparatuses and the separation apparatuses are each disposed in different reactor planes.

In this embodiment, the phase separation apparatus is configured as spatially separate apparatuses that are mutually fluid-connected. In particular, the condensation apparatus is configured as a condenser which is operated, for example, with cooling water as cooling medium. In addition, the separation apparatus is especially configured as a gas-liquid separator in which the liquid reaction product is separated from the gaseous residual gas mixture.

In this connection, a preferred embodiment of the reactor according to the invention is characterized in that the reactor has a first end and a second end, with condensation apparatuses and separation apparatuses in the reactor planes arranged spatially according to the following sequence from the first end in the direction of the second end: condensation apparatuses, separation apparatuses.

If the phase separation apparatuses are configured as separate condensation apparatuses and separation apparatuses, the reactor will thus comprise a total of four reactor planes, where the sequence of the arrangement of the reactor cell elements per reactor cell from the first then to the second end is as follows: reaction apparatus, cooling-down apparatus, condensation apparatus, separation apparatus.

A preferred embodiment of the reactor according to the invention is characterized in that the overall reactor is in a vertical arrangement with respect to the perpendicular imparted by gravity and the first end is at the top and the second end at the bottom.

A preferred embodiment of the reactor according to the invention is characterized in that the reaction apparatuses are disposed in an uppermost reactor plane, the phase separation apparatuses are disposed in a lowermost reactor plane, and the cooling-down apparatuses are disposed in a reactor plane between the uppermost and lowermost reactor planes.

If the phase separation apparatuses are configured as separate condensation apparatuses and separation apparatuses, the reaction apparatuses are disposed in an uppermost reactor plane, the separation apparatuses are disposed in a lowermost reactor plane, the cooling-down apparatuses are disposed beneath the reaction apparatuses, and the condensation apparatuses are disposed above the separation apparatuses.

A preferred embodiment of the reactor according to the invention is characterized in that the cooling-down apparatus is configured as a gas-gas heat exchanger, and where the gas-gas heat exchanger is configured such that the product gas exiting from the reaction apparatus is cooled down in the gas-gas heat exchanger against a stream of the feed gas mixture or against a stream of the residual gas mixture, especially by gravity.

The gas-gas heat exchanger is especially configured as an indirect heat exchanger.

In this embodiment, the thermal integration of the reactor is optimized in that the feed gas mixture or residual gas mixture to be converted over the solid catalyst is preheated by the stream of the product gas mixture exiting from the respective reaction apparatus. Since the residual gas mixture in the phase separation apparatus is cooled down further in each reaction cell after the cooling-down in the cooling-down stage, especially in the condensation apparatus of a phase separation apparatus, this cooled residual gas mixture can be used again as cooling medium in a downstream reaction cell for cooling-down of the product gas mixture exiting from this downstream reaction cell.

A preferred embodiment of the reactor according to the invention is consequently characterized in that the reactor is configured such that the feed gas mixture enters the reactor via the gas-gas heat exchanger of the first reaction cell, and is heated as it does so by the product gas mixture exiting from the reaction apparatus of the first reactor cell.

The gas-gas heat exchanger is preferably configured here such that the stream of the product gas mixture flows from the top downward within the gas-gas heat exchanger and the stream of the feed gas mixture or of the residual gas mixture flows from the bottom upward within the gas-gas heat exchanger.

In general, the vertical arrangement of the reactor cells (reaction apparatuses at the top, apparatuses for cooling and condensing further down) and the utilization of the countercurrent principle achieve the advantages that follow. Firstly, optimized heat transfer is achieved between the process streams through the utilization of the countercurrent principle. Heating in upward direction also achieves the highest temperature at the top of the reactor, i.e. where the reaction of the feed gas mixture is to be effected. At the same time, cooling is effected in downward direction, such that the temperatures are at their lowest in the bottom region of the reactor, where the condensation of the liquid reaction product is to be effected. This automatically and advantageously also leads to condensing in downward direction, which enables a natural condensate flow direction.

A preferred embodiment of the reactor according to the invention is characterized in that the gas-gas heat exchanger is configured as a thermoplate heat transferrer, and the thermoplate heat transferrer is configured such that the feed gas mixture or residual gas mixture flows through the thermoplate heat transferrer within the thermoplates and the product stream flows through the thermoplate heat transferrer between the thermoplates.

Thermoplate heat exchangers are alternatively also referred to as pillow plate heat transferrers. Correspondingly, the thermoplates may also be referred to as pillow plates. The thermoplates are especially arranged parallel to one another. The thermoplates are pillow plates through which material can flow. Between two thermoplates that are especially arranged parallel to one another, there is an interspace through which material can flow.

Thermoplate heat transferrers enable a particularly space-saving arrangement of the gas-gas heat exchanger within the reactor. In addition, the use of thermoplate heat transferrers results in advantages with regard to pressure drops on the process side. The use of thermoplates makes it possible in principle to achieve low pressure drops with simultaneously high heat transfer coefficients compared to conventional plate heat transferrers or shell-and-tube heat transferrers.

A preferred embodiment of the reactor according to the invention is therefore characterized in that the reaction apparatus is configured as a thermoplate heat transferrer, and the thermoplate heat transferrer is configured such that the cooling medium from the reaction apparatus flows through the thermoplate heat transferrer within the thermoplates and the solid catalyst is disposed between the thermoplates of the thermoplate heat transferrer, such that the feed gas mixture flows through the thermoplate heat transferrer between the thermoplates.

A further preferred embodiment of the reactor according to the invention is therefore characterized in that the condensation apparatus is configured as a thermoplate heat transferrer, and the thermoplate heat transferrer is configured such that the cooling medium of the condensation apparatus flows through the thermoplate heat transferrer within the thermoplates, and the product gas mixture to be condensed and the residual gas mixture flow through the thermoplate heat transferrer between the thermoplates.

In a preferred embodiment, the reaction apparatus is configured in accordance with the above configuration as a thermoplate heat transferrer, and/or the cooling-down apparatus, especially the gas-gas heat exchanger, according to the above configuration is configured as a thermoplate heat transferrer, and/or the condensation apparatus is configured according to the above configuration as a thermoplate heat transferrer.

In this case, preferably at least two series-connected reactor cell elements within a reactor cell are configured as thermoplate heat transferrers, for example the reaction apparatus and the cooling-down apparatus. It is also possible that all reactor cell elements are configured as thermoplate heat transferrers where this is possible, for example the reaction apparatus, the cooling-down apparatus and the condensation apparatus. In the case of corresponding arrangement of the thermoplate heat transferrers, this results in flow pathways in a line within each reactor cell, and in the best case in relation to the preheating of the feed gas mixture or residual gas mixture, the actual reaction, and the cooling-down and condensation of the product gas mixture. This further reduces flow losses and flow resistances, which also further minimizes pressure drop. In spite of a compact reactor design, there are thus only few deflections of the gaseous and liquid reaction media required.

The reaction apparatuses each have a first end and a second end, and are configured such that the feed gas mixture and/or the residual gas mixture enters the respective reaction apparatus at the first end in each case and exits from the respective reaction apparatus at the second end in each case. The cooling apparatus is preferably configured such that an inlet port of the cooling apparatus is disposed in a region of the first and/or in a region of the second end of the reaction apparatuses. If the inlet port of the cooling apparatus is disposed in a region of the first end of the reaction apparatuses, the cooling medium preferably enters into a heat-exchanging relationship with the solid catalyst only at the second end of a respective reaction apparatus. In other words, the cooling medium in this embodiment first flows from the first end of the reaction apparatus toward the second end thereof without cooling of the reaction mixture or control of the temperature thereof by heat transfer. The cooling apparatus in this case thus has fluid connection between the inlet port and the second end of the respective reaction apparatus which is not in a heat-exchanging relationship with the solid catalyst.

The cooling medium may have a higher temperature at the outlet port than at the inlet port. In particular, the cooling medium enters the reactor as boiling boiler feed water at the inlet port and exits again as steam at the outlet port.

The cooling apparatus optionally has a header system for the feeding of the cooling medium and a collector system for the removal of the heated cooling medium. The header has the function of distributing fresh cooling medium, especially boiling boiler feed water, after entry via the inlet port between the respective parts of the cooling apparatus for the reaction apparatuses in the different reactor cells. The collector system is assigned the function of combining heated cooling medium, especially steam, after exit from the respective parts of the cooling apparatus for the reaction apparatuses in the different reactor cells. The combined steam then exits from the reactor via the exit port of the cooling apparatus.

A preferred embodiment of the reactor according to the invention is characterized in that the reaction apparatus of a reactor cell has a multitude of compartments, where a first compartment is not filled with catalyst and is configured such that the feed gas mixture or residual gas mixture can flow through it from the bottom upward, and where compartments disposed downstream of the first compartment are filled with catalyst and are configured such that the feed gas mixture or residual gas mixture can flow through them from the top downward.

The reaction apparatus of a reactor cell preferably has a multitude of, i.e. at least two, spatially separated compartments. The compartment through which the flow passes first in gas flow direction, i.e. the first compartment, is preferably not filled with catalyst. This compartment serves primarily to transport the feed gas mixture or residual gas mixture preheated in the cooling-down apparatus, especially in the gas-gas heat exchanger, to the top of the reactor, especially to the top of the respective reaction apparatus. After deflection of this as yet unconverted stream of gas mixture or residual gas mixture, this flows through the reactor bed of the solid catalyst preferably from the top downward, in the case of vertical arrangement of the reactor. The flow passes here through the compartment downstream of the first compartment in gas flow direction or, if there are more than two compartments, through the component downstream of the first compartment in gas flow direction, in the same direction of the feed gas mixture or residual gas mixture, with conversion of the gas mixture or residual gas mixture over the solid catalyst to product gas mixture. At the same time, the gas mixture heated up by the exothermic reaction is cooled in countercurrent by the cooling medium of the respective reaction stage, especially boiling boiler feed water. The steam that forms on the cooling side of the reaction stage flows upward in the case of vertical arrangement of the reactor, which enables a natural steam flow direction. If the first compartment were also filled with solid catalyst, the cooling of the product gas mixture would proceed in cocurrent in the first compartment, which does not allow efficient cooling. Or the flow direction of the cooling medium would have to be reversed for the first compartment, which would unnecessarily complicate the design of the reactor.

In a further aspect of the invention, the use of the reactor according to the invention as per any of the above embodiments for production of methanol from synthesis gas is proposed, wherein the synthesis gas includes carbon oxides (CO, CO₂) and hydrogen (H₂).

In one embodiment of the use according to the invention, the synthesis gas does not include any carbon monoxide or carbon dioxide.

With regard to the reduction of carbon dioxide emissions, carbon monoxide-free synthesis gases in particular will become ever more important in future. Synthesis gas mixtures for production of methanol that contain carbon dioxide and hydrogen as the main component are possible, for example, by mixing carbon dioxide from industrial offgases, for example waste-to-energy plants, and climate-neutral hydrogen (called “blue” hydrogen). Climate-neutral hydrogen can be produced, for example, by electrolysis of water via climate-neutral power, for example from wind power.

### BRIEF DESCRIPTION OF THE DRAWINGS

The invention is elucidated in detail hereinafter by a working example. In the following detailed description reference is made to the accompanying figures which form a part of the working example and which contains an illustrative representation of a specific embodiment of the invention. In this connection, direction-specific terminology such as “top”, “bottom”, “front”, “back”, etc., is used with reference to the orientation of the described figure. Since components of embodiments may be positioned in a multiplicity of orientations, the direction-specific terminology is used for illustration and is in no way limiting. A person skilled in the art will appreciate that other embodiments may be used and structural or logical changes may be undertaken without departing from the scope of protection of the invention. The following detailed description is therefore not to be understood in a limiting sense, and the scope of protection of the embodiments is defined by the accompanying claims. Unless stated otherwise, the drawings are not necessarily true to scale.

In the description that follows and in the drawings, identical elements are identified by the same reference numerals. Arrows, if they are not provided with reference signs, illustrate the flow direction of the feed gas mixture, product gas mixture, residual gas mixture, liquid reaction product or combinations thereof within the reactor according to the invention and wherein:

FIG. 1 is a schematic diagram of one embodiment of the reactor according to the invention, and

FIG. 2 is a schematic representation of an example of a mechanical configuration of the reactor according to the invention.

### DETAILED DESCRIPTION OF PREFERRED EMBODIMENTS

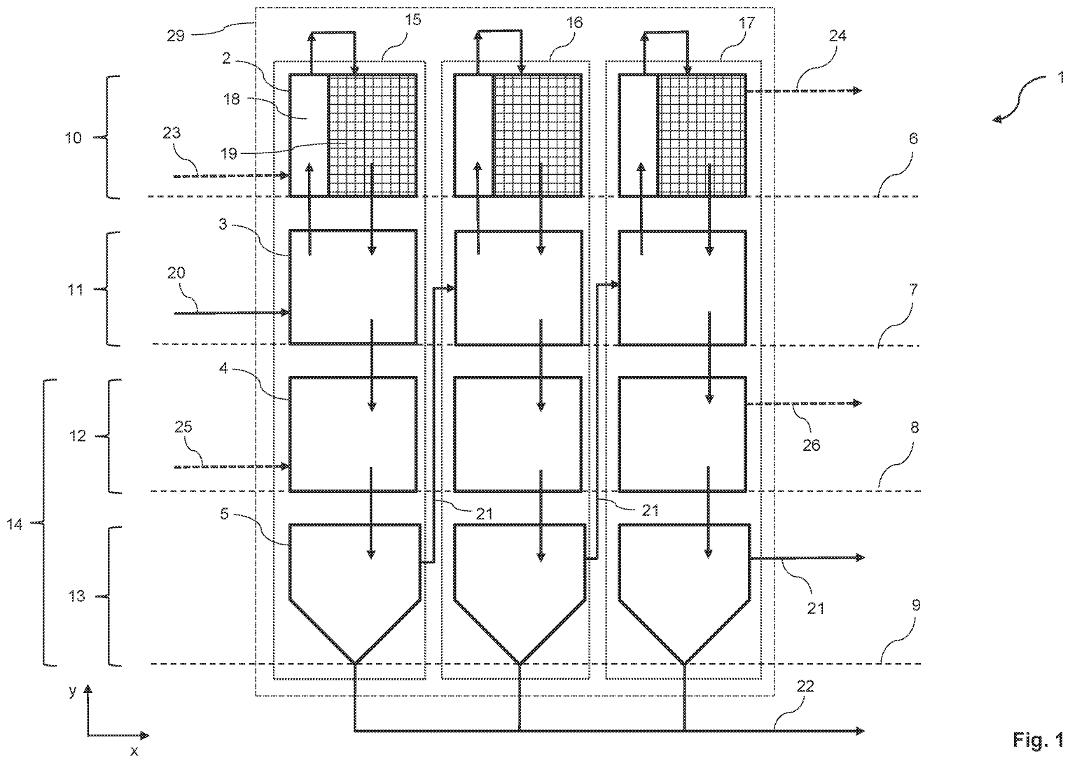

FIG. 1 shows a greatly simplified schematic illustration (schematic diagram) of one embodiment of the reactor according to the invention, which is to be used to elucidate, in particular, the arrangement of the individual reactor elements and the flow profile within the reactor.

The reactor 1 has a reactor shell 29 that surrounds all the essential reactor cell elements of the reactor 1. Feeds and drains or ports for these may extend through the reactor shell.

The reactor 1 according to FIG. 1 is suitable for production of methanol from synthesis gas as feed gas. Synthesis gas in the example is a mixture of hydrogen, carbon monoxide and carbon dioxide. The conversion of the feed gas over the solid catalyst in reactor 1 gives rise to a product gas mixture and a residual gas mixture. The product gas mixture contains methanol and water, and the residual gas mixture contains unconverted synthesis gas. The product gas mixture may further contain condensable by-products, for example higher alcohols, ketones, ethers and esters. The residual gas mixture may contain uncondensable by-products and unconvertible constituents from the crude synthesis gas, for example methane and nitrogen.

The reactor 1 comprises a total of three reactor cells which, according to FIG. 1, should be referred to as first reactor cell 15, second reactor cell 16 and third reactor cell 17. Each of the reactor cells has four different reactor cell elements. The reactor cell elements for each reactor cell 15, 16 or 17 are, in the arrangement from the top downward in the drawing, a reaction apparatus 2, a cooling-down apparatus 3, a condensation apparatus 4 and a separation apparatus 5. The reactor 1 has four reactor planes arranged parallel to one another. In the arrangement from the top downward as shown in the drawing, these are the reactor planes 6, 7, 8 and 9. Reactor cell elements of the same kind are each arranged in a common or the same reactor plane. Thus disposed in the first reactor plane are a total of three reaction apparatuses 2, which are described collectively as reaction apparatuses 10 in the first reactor plane 6. Also disposed in the second reactor plane are a total of three cooling-down apparatuses 3, which are described collectively as cooling-down apparatuses 11 in the second reactor plane 7. Also disposed in the third reactor plane are a total of three condensation apparatuses 4, which are described collectively as condensation apparatuses 12 in the third reactor plane 8. Also disposed in the fourth reactor plane are a total of three separation apparatuses 5, which are described collectively as separation apparatuses 13 in the fourth reactor plane 9. The condensation apparatuses 12 and separation apparatuses 13 may also be configured as integrated phase separation apparatuses, which are then disposed as phase separation apparatuses 14 in what is then a third reactor plane, in which case this third reactor plane would correspond to the reactor plane 9 in the diagram in FIG. 1. The reactor planes 6, 7, 8 and 9 should not be regarded as physical planes in the embodiment described, but merely as virtual planes. In other words, the reactor cell elements of the same kind are arranged in a line on this plane. For example, in the case of vertical arrangement of the reactor 1, the reactor cell elements of the same kind, by virtue of arrangement in the same reactor plane, are each disposed at the same height or essentially at the same height. If the principal axis of the reactor 1, as shown in FIG. 1, runs along the y axis in a Cartesian coordinate system, the extent of a reactor plane 6, 7, 8 or 9 is defined by means of an area that extends in x-z direction. The z axis runs perpendicular to the plane of the drawing shown. The positioning of the reactor planes 6, 7, 8 or 9 that extend in x-z direction, for example the height thereof, is defined by the position thereof on the y axis, i.e. at the position of the principal axis of the reactor.

The inventive arrangement of the reactor cell elements illustrated achieves the effect that reactor cell elements of different kinds that simultaneously have large differences in temperature in operation are not disposed alongside one another. In the case of a purely linear arrangement of all three reactor cells 15, 16 and 17 with vertical or horizontal alignment of the principal axis, the reaction apparatus 2 of the second cell 16 would, for example, be disposed alongside separation apparatus 5 of the first reaction cell 15. This would require high expenditure for the thermal insulation at least of one of the reactor cell elements, since the waste heat from the reaction apparatus would otherwise impair the performance and hence product yield of the separation apparatus. Moreover, there would be a high resultant degree of material stress expected between the hot reaction apparatus and the cold separation apparatus, which are fluid-connected to one another and hence automatically connected to one another via conduits.

The cooling-down apparatuses 11 are configured as gas-gas heat exchanger in the example according to FIG. 1. The cooling-down apparatus 3 of the first reactor cell 15 has a gas inlet 20 the introduction of the fresh synthesis gas. In the first cooling-down apparatus 3 of the first reactor cell 15, the feed gas is heated up by indirect heat exchange against the product gas mixture and residual gas mixture exiting from the reaction apparatus 2, with cooling-down of the product gas mixture and residual gas mixture in the cooling-down apparatus. After the feed gas mixture has been heated up in the cooling-down apparatus 3 of the first reactor cell 15, it enters the reaction apparatus 2. The reaction apparatus 2, like all reaction apparatuses 10 in the first reactor plane 6, contains a solid catalyst based on copper, for example, for conversion of the feed gas mixture to methanol and water. Each of the reaction apparatuses 2 additionally has at least two different compartments 18 and 19, where the first compartment 18 which is first in the arrangement in flow direction does not contain any catalyst, and hence no reaction takes place in the first compartment 18. The compartment which is at least second in the arrangement in flow direction, the further compartment 19 in the case shown, is filled with the solid catalyst. The reaction of the feed gas mixture to give water and methanol thus takes place in the further compartments 19. The solid catalyst in the further compartments 19 is in a heat-exchanging relationship with a cooling apparatus of the reaction apparatus 2, which is operated with boiling boiler feed water as cooling medium. The cooling apparatus serves primarily to control the temperature of the exothermic reaction, especially for compliance with a maximum permissible temperature in the catalyst bed of the solid catalyst. The reaction apparatus 2 therefore has a boiler feed water inlet 23. The incoming boiler feed water cools the product gas mixture and residual gas mixtures that have been heated up by the exothermic reaction in indirect heat exchange by the principle of evaporative cooling. The boiling boiler feed water flows from the bottom upwards in the reaction apparatus 2. Feed gas mixture flows from the top downward in the first compartment 18, i.e. in the same flow direction as the cooling medium, although no cooling of the feed gas takes place in the first compartment 18. The feed gas is subsequently deflected and then flows from the top downward in the further compartments 19 filled with catalyst in the reaction apparatus 2, while being cooled indirectly with boiling boiler feed water conducted in countercurrent thereto. The flow regime within the reactor shell 29 of the reactor 1 is configured such that boiler feed water flows from the bottom upward in each of the reaction apparatuses 2 and leaves them as saturated steam at the upper end of each reaction apparatus 2. The inventive reactor 1 has a corresponding header and collector system that enables this flow regime. In each of the reaction apparatuses 2, the product gas mixture and residual gas mixture that have been heated up over the catalyst are cooled indirectly in countercurrent with boiling boiler feed water. The heated-up cooling medium collected by the collector, the saturated steam, leaves the reactor at the saturated steam outlet 24 and can be utilized as export steam or within the process, for example in an upstream steam reformer.

The gas mixture and the unconverted gas mixture, i.e. residual mixture, subsequently enter the cooling-down apparatus 3 of the first reactor cell 15, where they are cooled down, as described above, in countercurrent against the gas mixture. There is still very substantially no condensation of the product gas mixture to the liquid reaction product here, but partial condensation in the cooling-down apparatus 3 is possible. The fully gaseous or partly condensed mixture then enters the condensation apparatus 4 in which the product gas mixture is fully or essentially fully condensed, such that liquid reaction product, a mixture of methanol and water and condensed unwanted by-products, and gaseous residual gas mixture are obtained in the condensation apparatus 4. The cooling in the condensation apparatus 4 of the first reactor cell 15 and the further reactor cells 16 and 17 is effected with cooling water by indirect heat exchange with the condensing mixture in countercurrent. The reactor 1 therefore has a cooling water inlet 25 and a cooling water outlet 26 in the condensation apparatuses 12 of the reactor plane 8. The reactor 1 also has, with regard to the cooling system in the condensation apparatuses 12, a corresponding header and collector system for the cooling water used for the condensation, such that fresh cooling water is available via a header (not shown) in each of the condensation apparatuses 4. Used cooling water is collected in the collector and discharged from the reactor 1 via the cooling water outlet 26.

The mixture of condensed liquid reaction product (methanol, water, condensed by-products) and gaseous residual gas mixture (water, carbon dioxide, carbon monoxide, methane, nitrogen, and condensed by-products) is separated in the separation apparatus 5 of the first reactor cell 15 into the corresponding liquid product portion and a gaseous portion. Separation apparatus 5 is configured, for example, as a gas-liquid separator. The liquid reaction product, essentially methanol and water, is discharged from the separation apparatus 5. The liquid reaction products from the separation apparatuses 13 in reactor plane 9 are combined and discharged from the reactor 1 as liquid reaction product 22. The liquid reaction products from each reactor cell can be combined within or outside the reactor shell 29, and this is done outside in the example according to FIG. 1. The liquid reaction product is then sent to the requisite workup in order to obtain pure methanol.

The separation apparatus 5 of the first reactor cell 15 has a residual gas mixture outlet 21. The residual gas mixture cooled down in the cooling-down apparatus 3 and condensation apparatus 4 is drawn off from the separation apparatus 5 via the residual gas mixture outlet 21 and introduced into the cooling-down apparatus 3 of the second reactor cell 16, where it is heated up by indirect heat exchange in accordance with the processes in the cooling-down apparatus 3 of the first reactor cell 15 in countercurrent against product gas mixture and residual gas mixture from the reaction apparatus 2 of the second reactor cell 16. The heated-up residual gas mixture then enters the first empty compartment of the reaction apparatus 2 of the second reactor cell 16, is deflected and is converted in the further compartments 19 of the reaction apparatus 2 of the second reactor cell 16 over the solid catalyst to give gas mixture and residual gas mixture. The further processes within the second reactor cell 16 and the third reactor cell 17 correspond very substantially to the above-described processes relating to the first reactor cell 15.

Synthesis gas unconverted in the third reactor cell 17, i.e. residual gas mixture, leaves the reactor at the (third) residual gas mixture outlet 21 of the separation apparatus 5 of that third reactor cell 17. Residual gas mixture drawn off at this residual gas mixture outlet 21 may be combined with the gas mixture in one embodiment and utilized further as such. In order to prevent the accumulation of inert constituents in the system, a proportion of the residual gas mixture drawn off from the third reactor cell may be removed as purge gas. The purge gas, in one example, may be used as fuel gas in an upstream steam reformer, or it is sent to a pressure swing adsorption apparatus for recovery of hydrogen.

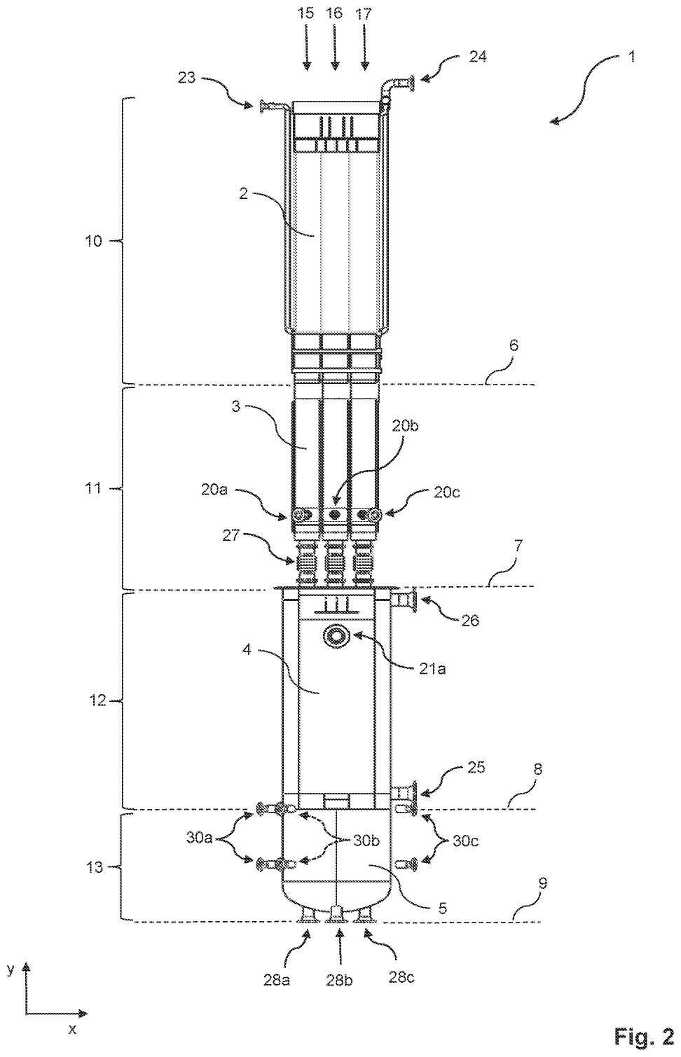

FIG. 2 shows an example of a mechanical configuration of the inventive reactor 1 according to FIG. 1. The reactor shell 29 is not shown here for the purposes of simplification and for reasons of clarity. In accordance with the diagram in FIG. 1, the reactor 1 according to FIG. 2 has a total of three reactor cells and four reactor planes. The reactor according to FIG. 2 has a principal axis along the y axis, which runs vertically here. From the top downward, in the reactor planes 6, 7, 8 and 9 that are arranged parallel to one another, there are accordingly respectively disposed three reaction apparatuses, three cooling-down apparatuses, three condensation apparatuses and three separation apparatuses. Correspondingly, the reaction apparatuses 10 are in the first reactor plane 6, the cooling-down apparatuses 11 are in the second reactor plane 7, the condensation apparatuses 12 are in the third reactor plane, and the separation apparatuses 13 are in the fourth reactor plane 9. The vertical arrangement of the principal axis of the reactor 1 enables a natural flow of condensate from the top downward, i.e. from the upper hotter parts of the reactor to the lower cooler parts of the reactor. The same applies to the flow of the cooling media to be heated up, especially the flow of the boiling boiler feed water. This enters the reactor 1 via the boiler feed water inlet 23 and exits from the reactor again as saturated steam via the saturated steam outlet 24. This heats up the boiler feed water within the reaction apparatus 10 from the bottom upward in each case. The same applies to the cooling water in the condensation apparatuses 12 that enters the reactor 1 at the cooling water inlet 25, flows from the bottom upward in each of the condensation apparatuses 12, and leaves the reactor 1 in heated form at the cooling water outlet 26.

The feed gas enters the reactor 1 via the feed gas mixture inlet 20a in the cooling-down apparatus 3 of the first reactor cell 15 and is converted in the reaction apparatus 2 of the first reactor cell 15, in accordance with the description relating to FIG. 1. The gas mixture is then cooled down in the cooling-down apparatus 2 of the first reactor cell 15, condensed in the condensation apparatus 4 of the first reactor cell 15, and separated as liquid reaction product in the separation apparatus 5 of the first reactor cell 15. Residual gas separated out in the separation apparatus 5 of the first reactor cell 15 exits via the residual gas mixture outlet 21a. It is then guided to the residual gas mixture inlet 20b via a pipeline either within the reactor shell 29 or outside the reactor shell 29.

The external guiding of the pipelines, i.e. outside the reactor shell 29, has the advantage that samples can easily be taken from the conduit for analytical purposes. In addition, this opens up the option of operating the three reactor cells 15, 16 and 17 in parallel. In such a mode of operation, the reactor according to the invention is used according to the principle of a one-stage synthesis. It would then be possible, however, to operate three reactors simultaneously, in which case these reactors here are each represented by the reactor cells 15, 16, 17.

The internal guiding of the pipelines, i.e. within the reactor shell 29, has the advantage that pipelines of shorter length overall are required, heat losses that inevitably occur are lower, and there are fewer potential opportunities for leaks.

Residual gas mixture exiting from the residual gas mixture outlet 21a then enters the cooling-down apparatus 3 of the second reactor cell 16 via the residual gas mixture inlet 20b, in order there in turn to cool product gas mixture first exiting from the reaction apparatus 2 of the reactor cell 16. This preheats the residual gas mixture, and it subsequently enters the reaction apparatus 2 of the second reactor cell 16 in preheated form at the top of the reactor cell 16. After conversion to the product gas mixture in reaction apparatus 2, cooling in cooling apparatus 3, condensation of the liquid reaction product in condensation apparatus 4 and finally separation in separation apparatus 5 of the second reactor cell 16, remaining residual gas mixture is drawn off from a further residual gas mixture outlet disposed at the height of the condensation apparatuses 12. This residual gas mixture outlet is disposed on the reverse side of the reactor 1 illustrated and is therefore not shown (according to the reference numeral system used, this residual gas mixture outlet would have had reference numeral 21b). The residual gas drawn off via that residual gas mixture outlet is introduced into the third reaction cell 17 via the residual gas mixture inlet 20c in accordance with the above statements. Residual gas separated out in the separation stage 5 of the third and last reactor cell 17 is finally drawn off via a last residual gas mixture outlet (likewise not shown, would correspond to residual gas mixture outlet 21c). This residual gas can optionally be conducted back to the feed gas mixture inlet 20a, i.e. is added to the feed gas mixture in this case.

Alternatively or additionally, the residual gas is sent to an apparatus for hydrogen recovery.

In a further option, it is also possible to add feed gas mixture to the respective residual gas mixtures before entry into the residual gas mixture inlets 20b and 20c.

The reactor 1 has one outlet for liquid reaction product per reactor cell. Shown correspondingly is the outlet 28a for the first reactor cell 15, the outlet 28b for the second reactor cell 16, and the outlet 28c for the third and last reactor cell 17. The outlets for the liquid reaction product 28a, 28b and 28c are disposed in the base region of the reactor 1 or in the base region of the separation apparatus 13. Each of the separation apparatuses 13 has two connection ports for control of the fill level of liquid reaction product in the respective separation apparatus 5. What are shown are the ports 30a of the separation apparatus 5 of the first reaction cell 15, the ports 30b of the separation apparatus 5 of the second reactor cell 16, and the ports 30c of the separation apparatus 5 of the third reactor cell 17.

For compensation of thermal stresses especially in longitudinal direction, each of the cooling-down apparatuses 11 has expansion bellows 27, each disposed upstream of the condensation apparatus that follows.

At least the reaction apparatuses 10 and the cooling-down apparatuses 11 of the reactor 1 are configured as thermoplate heat transferrers (pillow plate heat transferrers). The feed gas mixture or residual gas mixture flows through the reaction apparatus between the individual thermoplates, i.e. a region in which the catalyst bed of the solid catalyst is also present. The boiling boiler feed water flows through the reaction apparatuses within the thermoplates or pillow plates. Correspondingly, the product gas mixture and residual gas mixture being cooled down flows through the cooling-down apparatuses 11 between the thermoplates, while the feed gas mixture or residual gas mixture being heated up flows within the thermoplates or pillow plates. This enables a linear arrangement of the individual reaction apparatuses and cooling-down apparatuses with regard to the spaces through which product gas mixture and residual gas mixture or cooling medium (boiler feed water, feed gas mixture or residual gas mixture) flow. This allows the reactor 1 advantageously to have a particularly compact design.

### LIST OF REFERENCE SYMBOLS

1 Reactor
2 Reaction apparatus
3 Cooling-down apparatus
4 Condensation apparatus
Separation apparatus
6 First reactor plane
7 Second reactor plane
8 Third reactor plane
9 Fourth reactor plane
10 Reaction apparatuses in the first reactor plane
11 Cooling-down apparatuses in the second reactor plane
12 Condensation apparatuses in the third reactor plane
13 Separation apparatuses in the fourth reactor plane
14 Phase separation apparatuses
15 First reactor cell
16 Second reactor cell
17 Third reactor cell
18 First empty compartment of the reaction apparatus
19 Further catalyst-filled compartments of the reaction apparatus
20a,b,c Feed gas mixture inlet or residual gas mixture inlet
21a Residual gas mixture outlet
22 Liquid reaction product
23 Boiler feed water inlet
24 Saturated steam outlet
25 Cooling water inlet
26 Cooling water outlet
27 Expansion bellows
28a,b,c Outlet for liquid reaction product
29 Reactor shell
30a,b,c Ports for fill level control

It will be understood that many additional changes in the details, materials, steps and arrangement of parts, which have been herein described in order to explain the nature of the invention, may be made by those skilled in the art within the principle and scope of the invention as expressed in the appended claims. Thus, the present invention is not intended to be limited to the specific embodiments in the examples given above.

What is claimed is:

1. A reactor for performing exothermic equilibrium reactions, in which a feed gas mixture and/or a residual gas mixture is/are at least partly convertible over a solid catalyst, wherein a product gas mixture and a residual gas mixture are obtainable, wherein the product gas mixture contains a liquid reaction product which is condensable at reaction pressure and below the reaction temperature, the reactor comprising:
a reactor shell and a multitude of series-connected and mutually fluid-connected reactor cells disposed within the reactor shell, wherein
each reactor cell comprises the following series-connected and mutually fluid-connected reactor cell elements:
a reaction apparatus configured for converting the feed gas mixture and/or the residual gas mixture to the product gas mixture, having the solid catalyst and a cooling apparatus which is in a heat-exchanging relationship with the solid catalyst and through which a cooling medium can flow;
a cooling-down apparatus configured for cooling down the product gas mixture exiting from the reaction apparatus;
a phase separation apparatus configured for condensing and separating the liquid reaction product from the cooled-down product gas mixture, where the condensed liquid phase contains the reaction product, and the gaseous phase the residual gas mixture;
and wherein each reactor cell comprises
a means of discharging the reaction product from the reactor cell, and
a means of discharging the residual gas mixture from the reactor cell, and
a means of feeding the residual gas mixture to a downstream reactor cell, if present;
and wherein the reactor has
a multitude of reactor planes disposed in a mutually parallel arrangement within the reactor shell, where reactor cell elements of the same kind are disposed in the same reactor plane.

2. The reactor according to claim 1, wherein exclusively one kind of reactor cell element is disposed in each of the reactor planes.

3. The reactor according to claim 1, wherein the number of reactor planes present corresponds at least to the number of types of reactor cell element present.

4. The reactor according to claim 1, wherein the reactor cells are in a vertical arrangement with respect to the perpendicular imparted by gravity, such that streams are conductable from the bottom upward and from the top downward within and between the reactor cell elements.

5. The reactor according to claim 1, wherein the reactor cell elements are arranged within the reactor shell according to a two-dimensional matrix with a number y of columns and a number x of rows, wherein the number y corresponds to columns of the number of reactor cells, and the number x corresponds to rows of the number of different types of reactor cell element, wherein one column in the matrix comprises one reactor cell of each of the different types of reactor cell element, and one row in the matrix comprises a reactor plane having a number of reactor cell elements of the same type.

6. The reactor according to claim 1, wherein the reactor planes are arranged parallel to one another and successively from the top downward.

7. The reactor according to claim 1, wherein the reactor has a first end and a second end, with reactor cell elements in the reactor planes arranged spatially according to the following sequence from the first end in the direction of the second end: reaction apparatus, cooling-down apparatus, phase separation apparatus.

8. The reactor according to claim 1, wherein each phase separation apparatus comprises a condensation apparatus and a separation apparatus, where the condensation apparatuses and the separation apparatuses are each disposed in different reactor planes.

9. The reactor according to claim 8, wherein the reactor has a first end and a second end, with condensation apparatuses and separation apparatuses in the reactor planes arranged spatially according to the following sequence from the first end in the direction of the second end: condensation apparatuses, separation apparatuses.

10. The reactor according to claim 7, wherein the overall reactor is in a vertical arrangement with respect to the perpendicular imparted by gravity and the first end is at the top and the second end at the bottom.

11. The reactor according to claim 1, wherein the reaction apparatuses are disposed in an uppermost reactor plane, the phase separation apparatuses are disposed in a lowermost reactor plane, and the cooling-down apparatuses are disposed in a reactor plane between the uppermost and lowermost reactor planes.

12. The reactor according to a claim 1, wherein the cooling-down apparatus is configured as a gas-gas heat exchanger, and where the gas-gas heat exchanger is configured such that the product gas exiting from the reaction apparatus is cooled down in the gas-gas heat exchanger against a stream of the feed gas mixture or against a stream of the residual gas mixture.

13. The reactor according to claim 12, wherein the gas-gas heat exchanger is configured such that the stream of the product gas mixture flows from the top downward within the gas-gas heat exchanger and the stream of the feed gas mixture or of the residual gas mixture flows from the bottom upward within the gas-gas heat exchanger.

14. The reactor according to claim 12, wherein the reactor is configured such that the feed gas mixture enters the reactor via the gas-gas heat exchanger of the first reaction cell, and is heated as it does so by the product gas mixture exiting from the reaction apparatus of the first reactor cell.

15. The reactor according to claim 12, wherein the gas-gas heat exchanger is configured as a thermoplate heat transferrer, and the thermoplate heat transferrer is configured such that the feed gas mixture or residual gas mixture flows through the thermoplate heat transferrer within the thermoplates and the product stream flows through the thermoplate heat transferrer between the thermoplates.

16. The reactor according to claim 1, wherein the reaction apparatus is configured as a thermoplate heat transferrer, and the thermoplate heat transferrer is configured such that the cooling medium from the reaction apparatus flows through the thermoplate heat transferrer within the thermoplates and the solid catalyst is disposed between the thermoplates of the thermoplate heat transferrer, such that the feed gas mixture flows through the thermoplate heat transferrer between the thermoplates.

17. The reactor according to claim 1, wherein the reaction apparatus of a reactor cell has a multitude of compartments, where a first compartment is not filled with catalyst and is configured such that the feed gas mixture or residual gas mixture can flow through it from the bottom upward, and where compartments disposed downstream of the first compartment are filled with catalyst and are configured such that the feed gas mixture or residual gas mixture can flow through them from the top downward.
